(19) United States
(12) Patent Application Publication
(10) Pub. No.: US 2010/0063358 A1
(43) Pub. Date: Mar. 11, 2010

(54) CHANNELED FLEXIBLE SLEEVE FOR MEDICAL ARTICLES

(76) Inventor: Fred Kessler, Mayfield Heights, OH (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/305,172

(22) PCT Filed: Jul. 9, 2007
(86) PCT No.: PCT/US2007/073079

§ 371 Date: Dec. 16, 2008

Related U.S. Application Data
(60) Provisional application No. 60/819,266, filed on Jul. 7, 2006.

(51) Int. Cl. A61B 1/00

(52) U.S. Cl. 600/121

(57) ABSTRACT

A medical tool includes a flexible sleeve placed over a flexible endoscope tube of an endoscope, with the flexible sleeve having one or more channels for providing additional access to the interior of a patient's body. Various tools may be inserted into the channels in order to perform surgical techniques within the body, such as within the gastrointestinal (GI) tract of the patient. Such insertable tools may include scalpels, scissors, or gripping tools. The tools may be flexible tools, and may include electrically-powered tools or non-electrically-powered tools.

### CROSS-REFERENCE TO RELATED APPLICATIONS

This application claims the benefit of U.S. Provisional Application No. 60/819,266 filed Jul. 7, 2006, which is hereby incorporated by reference in its entirety.

### BACKGROUND OF THE INVENTION

1. Field of the Invention

The invention is in the general field of medical tools, and more specifically relates to tools and methods involving endoscopes.

2. Description of the Related Art

An endoscope is a tubular medical tool used for imaging and/or performing actions inside the body of a patient, such as taking biopsies and retrieving foreign objects. Some endoscopes provide an additional channel to allow entry of medical instruments or manipulators into the body of the patient. Modern endoscopes may be rigid or be generally flexible, and may be utilized for diagnostic and/or treatment purposes in many parts of the body, such as in the gastrointestinal (GI) tract. For example, gastroscopy involves using an endoscope to examine and/or perform actions along the lining of the esophagus, stomach, or the duodenum. Gastroscopy is often used to diagnose and/or treat ulcers and other sources of bleeding, or to guide biopsy of suspected gastrointestinal cancers. Another type of endoscopy is colonoscopy, which is the examination of the inside of the colon and large intestine, such as for diagnostic purposes.

Many endoscopic procedures are minimally invasive and cause only mild discomfort or pain, wherein only a topical anaesthesia is necessary. For these reasons, some patients prefer to undergo endoscopic surgery rather than other more invasive surgical procedures in order to reduce pain and time for recovery. Accordingly, the number of endoscopic surgeries is increasing, and doctors are attempting to perform increasingly complex procedures through the use of endoscopes. The result of complex endoscopic surgeries is that doctors must use a myriad of medical instruments wherein each instrument must be inserted into and removed from the additional channel of the endoscope.

This process of repeatedly inserting and removing the instruments is not only more time consuming for the physician, but also more risky for the patient. Further, endoscopic instruments are costly to replace, and thus some doctors prefer to maintain their existing endoscopic instruments, thereby allowing them to monetize their existing equipment and also saving them the time and energy necessary to learn the operations of a new endoscope.

### SUMMARY OF THE INVENTION

Embodiments of the present invention relate to endoscopic instruments or similar tools used for imaging and/or performing actions inside the body of a patient, such as, for example, taking biopsies, cutting and/or suturing tissue, cauterizing, and retrieving objects. Various embodiments of the present invention can address some or all of the noted shortcomings associated with existing endoscopes. For example, and in accordance with one aspect of the present invention, a flexible channeled sleeve is provided to substantially surround or encapsulate at least a portion of an endoscope. The sleeve is preferably adapted to extend at least as long as the working length of the endoscope. Further, the sleeve is preferably configured to be attached to and work along side existing endoscopes.

The sleeve attaches to the endoscope, preferably in a releasable manner, such that the sleeve does not slip up or down the endoscope to any substantial extent once the sleeve has been secured to the endoscope. The sleeve also comprises at least one outer member that defines a channel along the endoscope to allow a medical instrument to be passed through the channel to the surgical site. Preferred embodiments of the channeled sleeve are single-use, disposable, convenient to use, and significantly improve the efficacy and safety of a wide variety of endoscopic procedures.

In accordance with another aspect of the present invention, a single-use, disposable sleeve is provided for use with an endoscope, which includes an elongated body having a working length that terminates at a distal end. The sleeve comprises a flexible elongated generally cylindrical hollow member having an inner size and a cross-section shape that generally matches the cross-section shape and size of the endoscope elongated body, at least along the working length, such that the endoscope fits within the hollow member. The hollow member has a wall thickness that is at least an order of magnitude smaller than the inner size of the hollow member and comprises a distal portion. The distal portion is configured for attachment to a distal portion of the endoscope to substantially secure the hollow member to the endoscope. At least one flexible elongated outer member is attached to the hollow member to define at least one elongated channel. The channel has a length that is at least substantially equal to the working length of the endoscope's elongated body, and has a sealed proximal end. The channel is sealed along it length and is sufficiently sized to receive a medical instrument.

### BRIEF DESCRIPTION OF THE DRAWINGS

The foregoing and other features, aspects and advantages of the present invention are described in detail below with reference to the drawings of preferred embodiments, which are intended to illustrate and not to limit the invention. The drawings comprise seventeen figures in which:

FIG. 1 is a top plan view of an endoscope device, which represents an exemplary medical device with which the present channeled sleeve can be used;

FIG. 2 is top plan view of the channeled sleeve device, which is configured in accordance with a preferred embodiment of the invention, as disposed on the endoscope of FIG. 1;

FIG. 3 is a top plan view of the channeled sleeve of FIG. 2;

FIG. 4A is a cross-sectional view of the endoscope/sleeve assembly taken along line 4-4 of FIG. 2, with an outer member of the channeled sleeve generally in pre-expanded state;

FIG. 4B is an enlarged cross-sectional view of the endoscope/sleeve assembly of FIG. 4A;

FIG. 5 is a cross-sectional view of the endoscope/sleeve assembly, similar to FIG. 4A, with the outer member in an expanded or state;

FIG. 6A is an enlarged view illustrating the hollow member of the channeled sleeve device of FIG. 2, with a releasable band strip surrounding the endoscope and the hollow member with open distal ends of the channels;

FIG. 6B is an enlarged view illustrating another embodiment of the channeled sleeve device having channels with initially closed distal ends;

FIG. 7A is an enlarged cross-sectional view of a split clip of the channeled sleeve device of FIG. 2;

FIG. 7B is a side elevational view of a split clip that is configured in accordance with another embodiment of the present invention;

FIG. 8 is a cross-sectional view of an embodiment of the channeled sleeve device with an inner hollow member and a fused outer member;

FIG. 9 is a cross-sectional view of another embodiment of the channeled sleeve device with multiple outer members fused to the inner hollow member;

FIG. 10 is a schematic illustration of a medical instrument being inserted into the channeled sleeve device of FIG. 2;

FIG. 11 is a schematic illustration of a medical instrument being inserted into the channeled sleeve device having a proximal port configured in accordance with another preferred embodiment of the invention;

FIG. 12 is an enlarged perspective view of a proximal port of the channeled sleeve device shown in FIG. 11, which is configured in accordance with a preferred embodiment of the present invention, for inserting medical instruments into the outer member of the channeled sleeve;

FIG. 13 is a cross-sectional view of an endoscope/sleeve assembly where the channeled sleeve is configured in accordance with another preferred embodiment of the invention and where an outer member thereof is an expanded state;

FIG. 14 is a top plan view of a channeled sleeve device configured in accordance with an additional preferred embodiment to have a tapered distal end;

FIG. 15 is a top plan view of the channeled sleeve member having a tapered distal end configured in accordance with a further embodiment of the present invention; and

FIG. 16 is a schematic illustration of the channeled sleeve device having perforations for adjusting the length of the sleeve, and being dispensable from a roll of like sleeves.

### DETAILED DESCRIPTION OF THE PREFERRED EMBODIMENT

Embodiments of the present invention provide a channeled sleeve configured for attachment to an endoscope, which improves a clinician's ability to utilize multiple medical instruments during an endoscopic surgical procedure in a safe and efficient manner. In more preferred embodiments, the sleeve is a single-use, disposable device. However, it will be appreciated that embodiments of the described channeled sleeve and/or features thereof may be used in a wide variety of applications (including multi-use applications).

The term “endoscope” includes but is not limited to elongated instruments used for imaging, diagnosing, and/or performing actions or treatments inside the body of a patient, such as taking biopsies and retrieval of foreign objects. In one preferred embodiment, the channeled sleeve is configured to be used with an endoscope and other medical instruments to perform a Natural Orifice Surgery (NOS), or more specifically a Natural Orifice Transluminal Endoscopic Surgery (NOTES). In either of the foregoing surgical procedures, the channeled sleeve is positioned on the endoscope, wherein the endoscope is positioned at the surgical site by passing through a natural orifice, for example, anus, ear, mouth, nose, urethra, vagina, or the like, and then through an internal incision in an area (for example, bladder, colon, ear canal, nostril, stomach, vagina, or the like) near the surgical site. However, many of the principles of the channeled sleeve described herein can also be used with other types of endoscopes or other surgical access/treatment devices, as well as with a wide variety of medical articles and/or instruments. For example, but without limitation, the channeled sleeve can be used with catheters, obturators, fluoroscopes, etc.

In many embodiments, the channeled sleeve can be a single-use, disposable item having a working length that is attached to an endoscope (or other elongated medical article) to provide at least one outer channel configured to allow an additional medical instrument access to the surgical site simultaneously with an instrument passing through the endoscope. In some embodiments, the channeled sleeve is integrally formed onto such devices, and in other embodiments, the channeled sleeve is attached, retro-fitted, or secured to such devices.

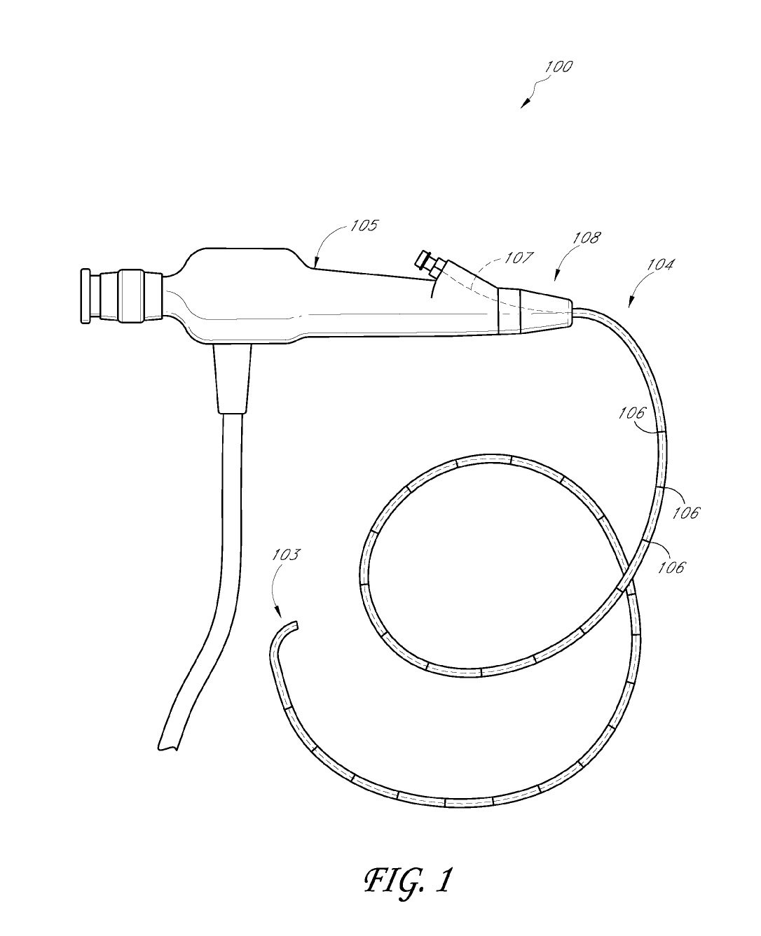

Thus, with reference to FIG. 1, there is illustrated an endoscope 100 with which the channeled sleeve can be used. As shown, the endoscope 100 comprises a distal portion 103, which is intended for insertion into the patient's body, and a handle or hand piece 105, which remains outside the patient's body. The hand piece 105 includes a distal end 108 from which an elongated body of the endoscope 100 extends. The endoscope 100 typically comprises an internal channel 107 for inserting medical instruments, and markers 106 that indicate the positioning of the endoscope 100 within the patient's body (i.e., the inserted length).

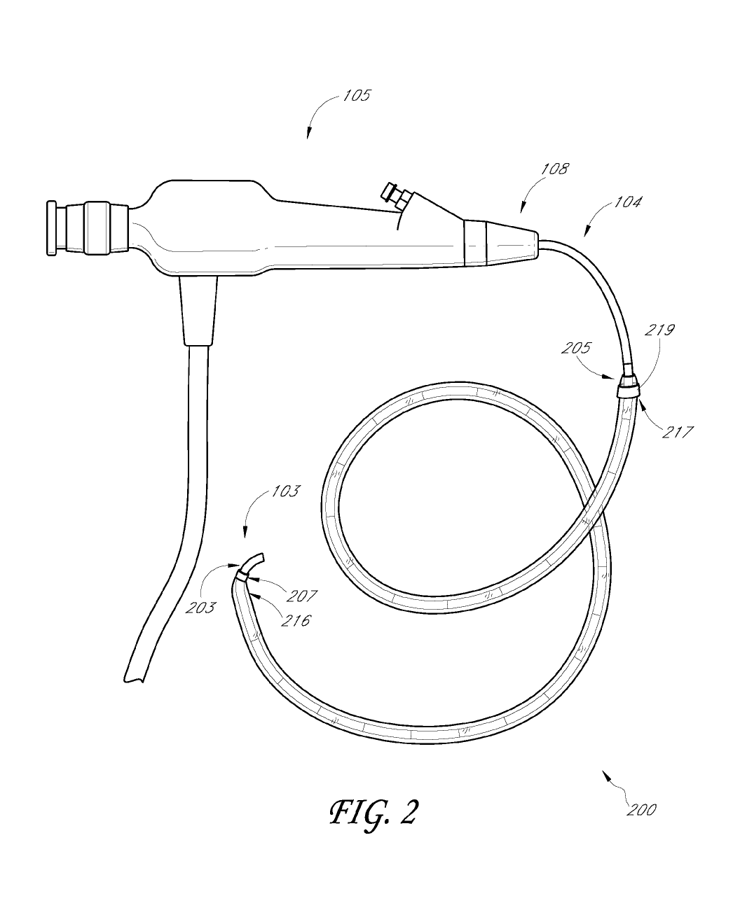

FIG. 2 illustrates a preferred embodiment of the channeled sleeve 200 as positioned on the endoscope 100. The sleeve 200 includes a distal end 203 and a proximal end 205. In use, the distal end of the endoscope is slid into the sleeve's proximal end 205 and is advanced through the sleeve until it emerges from the sleeve's distal end 203.

The channel sleeve 200 includes an inner hollow member 201 that defines a channel, lumen, guide, or the like therein. In the illustrated embodiment, the hollow member 201 has a lumen with an inner size (e.g., a diameter) that substantially matches the outer size (e.g., an outer diameter) of the endoscope 100. In some applications, however, the inner size can be slightly larger or smaller than the elongated body onto which the sleeve is disposed. The lumen of the hollow member 201 also has a cross-section shape that at least generally matches the cross-section shape of the endoscope 100.

The hollow member 201 has a length that is at least as long as the working length of the endoscope 200, i.e., the length of the endoscope inserted into the patient in a given procedure. In some embodiments, as described below, the length of the sleeve 200 can be adjusted to suit a particular application. For example, where less of the instrument is inserted into the patient's body, due to the size of the patient, the working length of the endoscope will be shorter. In such applications, though, the hollow member 201 should have a length at least as long as the inserted portion of the instrument.

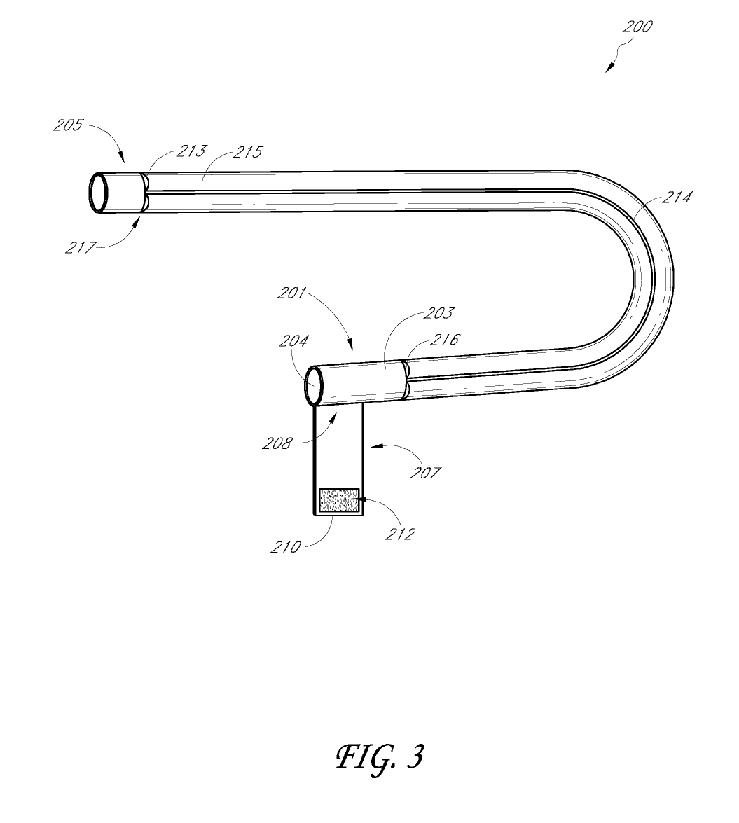

The hollow member 201 comprises an interior surface 204 (shown in FIG. 3) on which a lubricant, a coating, or the like, preferably is applied to or is integral with the surface 204 to facilitate the axial movement of the endoscope 100 through the hollow member 201. In some preferred embodiments, the lubricant is silicon, mineral oils, KY jelly products, or the like. In some preferred embodiments, the coating is Teflon® or other coating that provides an impermeable low friction surface. In other embodiments, the endoscope is treated with a lubricant prior to inserting into the channel sleeve 200. In such embodiments, the sleeve 200 can include a lubricated inner surface as well.

At least the distal end of the channel sleeve 200 is attached to the endoscope 100 at a point near the distal end 103 of the endoscope 100. Preferably, a seal is also formed between the channel sleeve 200 and the endoscope 100 to inhibit fluids, gases and other materials from escaping the surgical field through the space between the endoscope 100 and the hollow member 201. In the illustrated embodiment, the distal portion 203 of the hollow member 201 comprises at least one band strip 207 for both of these purposes. The band strip 207 is preferably releasable, but need not be so. Also, while it is preferred that one mechanism be used to attach the sleeve 200 to the endoscope 100 and to form the seal, separate mechanisms can be used in other embodiments to perform each of these functions.

As seen in FIG. 3, the band strip 207 has a base portion 208 that is attached to the distal portion 203 of the hollow member 201. In this preferred embodiment, the band strip 207 is integrally formed with the hollow member 201, although in other embodiments it can be fastened to the hollow member (e.g., adhered by a biocompatible adhesive). The band strip 207 extends away from the longitudinal axis of the hollow member, preferably at about 90°, although it can extend at other angles relative to the longitudinal axis. The band strip 207 terminates at an outer end portion 210.

The outer end portion 210 includes a fastener 212 to couple the outer end 210 to at least one of: (1) a portion of the band strip 207; (2) a portion of the hollow member 201; or (3) a portion of the endoscope 100. In the illustrated embodiment, the fastener 212 comprises an adhesive patch 212 that is configured to adhere to the surface of the band strip 207. Although not shown, the adhesive patch 212 preferably is covered with a release liner before the channeled sleeve 200 is slipped onto the endoscope. Additionally, the fastener can take other forms, such as, for example, but without limitation, a snapping, latching or hook/loop device.

The band strip 207 is used to secure the hollow member 201 onto the distal end 103 of the endoscope 100 by wrapping the band strip 207 around the hollow member 201, thereby preferably forming a seal between the hollow member 201 and the endoscope 100 such that fluid and other materials are prevented from entering into the hollow member 201. The adhesive patch 212 preferably adheres onto a portion of itself, which has been tightly wrapped about the endoscope 100 and the hollow member 201. In some embodiments, the adhesive may be selected to be somewhat releasable to facilitate removal of the sleeve 200 from the endoscope 100. In other embodiments, the adhesive can form at least a semi-permanent joint, in which case the band 207 and/or hollow member 201 can be cut to remove the sleeve 200 from the endoscope 100.

The band strip 207 is constructed of any flexible material, such as plastic, polymer, or the like, and may have at least one textured surface for engaging the hollow member 201 so as to grip onto the hollow member 201. The band strip 207 in the illustrated embodiment is formed of the same flexible materials of which the hollow member 201 is made. In other embodiments, the band strip 207 can be formed of other flexible material that may or may not be substantially elastic (e.g., a stretchable band) or the band strip 207 can be a string or like material suitable for tying or securing the hollow member 201 onto the distal end 103 of the endoscope 100.

In this preferred embodiment, the hollow member 201 lacks any significant columnar strength and/or radial strength such that the hollow member 201 will flex, bend and collapse when a force, which is sufficient to advance the endoscope into a patient's body, is applied. The hollow member 201 is configured to neither translate axial forces along the length of the hollow member 201 nor exert a radial force on surrounding body tissue. Additionally, the hollow member 201 is compliant both longitudinally and radially.

The thickness of the wall of the hollow member 201 can be at least an order of magnitude smaller in size than the inner size (e.g., diameter) of the hollow member 201. In some embodiments, the wall thickness of the hollow member 201 is at least two to five orders of magnitude smaller in size than the diameter of the hollow member 201. In the illustrated embodiment, the diameter of the hollow member 201 is about 20 mm; however, in other embodiments, the diameter of the hollow member 201 can range between 1 mm and 20 mm, or be even smaller. The wall thickness of the hollow member 201 is preferably thin relative to the diameter of the hollow member (e.g., approximately 0.02 mm) so as to only minimally increase the profile of the endoscope device and to not affect the flexibility of the endoscope 100 by any meaningful degree. The thickness of the hollow member 201 can be less than 2 mm in some embodiments, preferably less than 0.5 mm, and more preferably is less than 0.1 mm (e.g., 0.03 mm).

One or more channels 213 of the sleeve 200 are created by one or more outer members 215 attached to the hollow member 201. In the illustrated embodiment, the sleeve 200 includes one outer member 215 having a length that is shorter than the length of the hollow member 201. In this manner, at least the distal end of the hollow member 201 remains exposed for attachment to the endoscope 100. In other embodiments, however, portions of the outer member's distal end can be fastened to the endoscope 100 (along with or without the distal end of the hollow member 201).

The outer member 215 preferably has a similar cross-sectional shape to that of the hollow member 201, although in some embodiments, the outer member 215 and the hollow member 201 can have differing cross-sectional shapes. The inner size of the outer member 215, in the illustrated embodiment, is also larger than that of the hollow member 210, but need not be in all embodiments as explained below.

The wall thickness of the outer member 215 is preferably thin relative to the diameter of the hollow member (e.g., approximately 0.02 mm) so as to only minimally increase the profile of the endoscope device and to not affect the flexibility of the endoscope 100 by any meaningful degree. The thickness of the outer member 215 can be less than 2 mm in some embodiments, preferably less than 0.5 mm, and more preferably is less than 0.1 mm (e.g., 0.03 mm).

In some preferred embodiments, the hollow member 210 and the outer members 215 are formed or extruded as one integrally formed device without any bonding joints or the like.

In other preferred embodiments, the outer member 215 is fastened to the hollow member 201 preferably at a number of locations. In the illustrated embodiment, as best seen in FIGS. 3-5, four longitudinally extending bonds 214 join the outer member 215 to the hollow member 201 to form four channels 213. Each channel 213 has a sufficient size to receive one or more instruments used in an endoscopic procedure (e.g., a NOTES procedure). While the bonds 214 preferably seal each channel 213 from the adjacent channels 213, the bonds 214 can be interrupted along their length to provide at least some fluidic communication between some or all of the channels 213. Such openings between the channels 213 may also provide different routing paths for instruments passed through other embodiments of the sleeve.

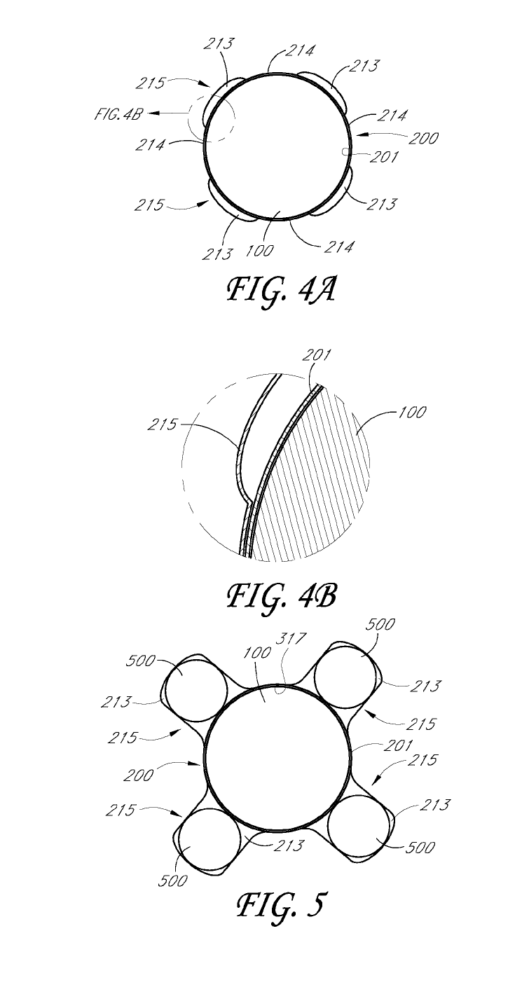

As seen in FIG. 4A, the channels 213 preferably lie in a generally collapsed state before use. FIG. 4B is an enlarged view of the inset area encircled in FIG. 4A. (FIGS. 4A and 4B exaggerate the opening degree of the channels 213 in the collapsed state to allow the channels 213 to be identified.) This configuration minimizes the profile of the sleeve 200 when it is attached to the endoscope 100 and is inserted into a patient's body. Once the distal end 103 of the endoscope 100 is positioned at a desired location within the patient's body, the physician can insert one or more instruments 500 into the channels 213 of the sleeve. Insertion of the instruments causes the channels to expand, as illustrated in FIG. 5. Such enlargement of the channels 213 can occur as a result of folded material unfolding to create the expanded channel space, and/or as a result of the material stretching. The channels 213 may also be enlarged by introducing a fluid (e.g., saline) into the channel, which might occur before the physician introduces an instrument 500 into the channel 213.

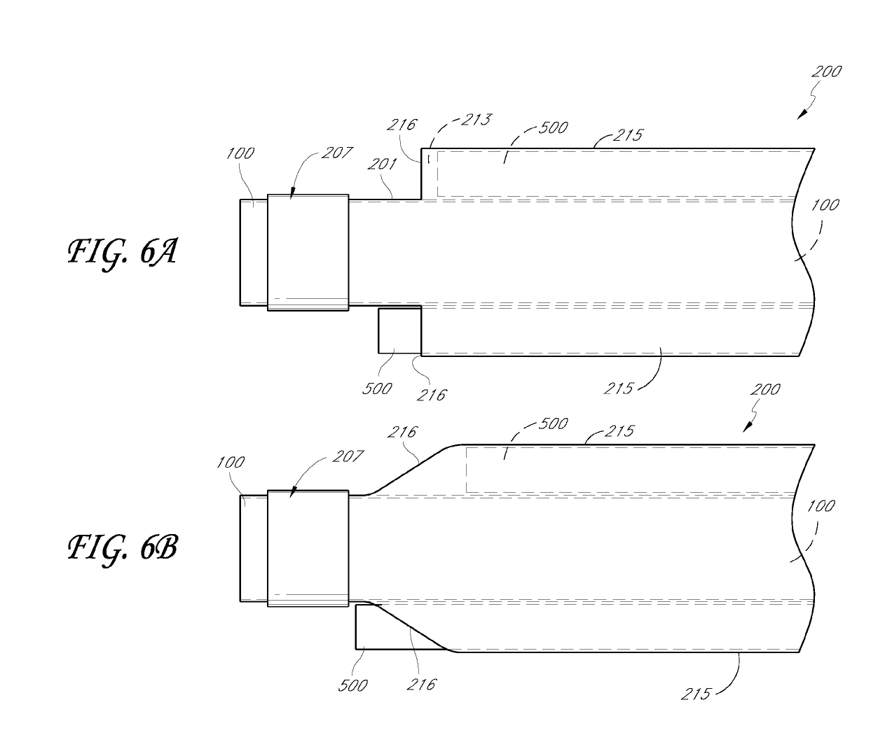

As best seen in FIG. 6A, the distal ends 216 of channels 213 are initially open, although in a collapsed state before being used. An instrument 500 or a fluid can thus freely pass through the open distal end 216 of the channel 213. In other embodiments, as illustrated in FIG. 6B, one or more of the distal ends 216 can be initially closed (for example by sealing at least a portion of the outer member's distal end to the distal end of the hollow member 201). Either a stylet or the instrument itself can be used to pierce the distal end 216 of the channel 213 to open the channel to the surgical site. In connection with embodiments having closed distal ends 216, a fluid (e.g., saline) can be used to form a gas seal, either during the insertion process or during a surgical procedure, before the channel 213 is used. Additionally, in some embodiments, the proximal ends 217 of channels 213 are configured to connect to a negative pressure or vacuum source that is readily available at the patient facility. Negative pressure or a vacuum can also be applied to the proximal ends 217 of channels 213 having closed distal ends 216 so as to collapse the channels 213. Collapsing channels 213 having open distal ends 216 by applying negative pressure or a vacuum is also possible in some circumstances.

In use, the proximal ends 217 of the channels 213 are also sealed to inhibit the flow of fluids from the body through the channels 213. In some embodiments, the proximal ends 217 are initially sealed and in other embodiments the proximal ends 217 are initially open. In the illustrated embodiment shown in FIG. 3, the proximal ends 217 are initially open.

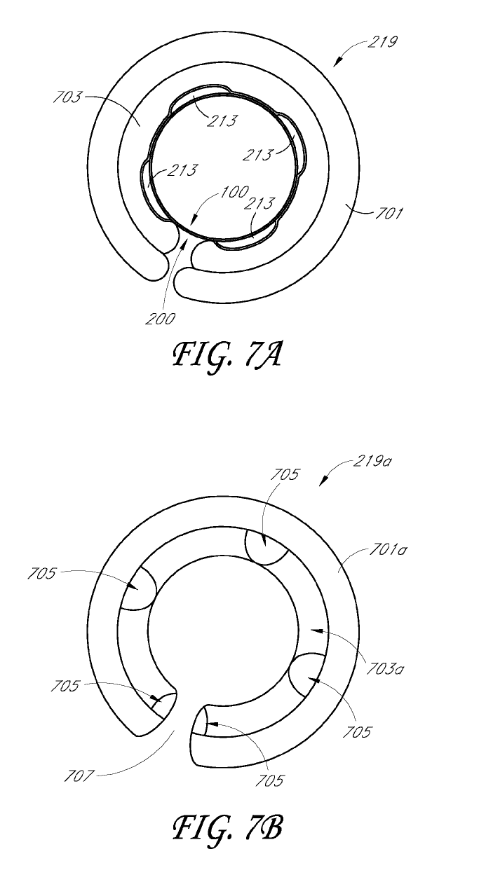

A sealing device is to be used to seal the proximal ends 217 of the channels 213 while allowing the introduction of an instrument 500. In the illustrated embodiment, the sealing device comprises a split clamp or a split clip 219, which is illustrated in FIG. 7A in a position about the endoscope 100 and the proximal end of the sleeve 200. (FIG. 7A, like FIG. 4, exaggerates the opening degree of the channels 213 in the collapsed state to allow the channels 213 to be identified.) The clip 219 surrounds all or substantially all of the circumference of the sleeve and applies an inward pressure to seal the proximal ends 217 of the channels 213.

The clip 219 in this preferred embodiment comprises a semi-rigid portion 701 that is sufficiently flexible to attach or snap-on to the endoscope 100 positioned within the sleeve 200. The split clip 219 further comprises a compressible portion 703 that is sandwiched between the semi-rigid portion 701 and the endoscope 100 thereby sealing the hollow member 201. The compressible portion 703 can be formed of materials which have a suitable elasticity and compressibility, including, but not limited to, Kraton® polymer compounds, such as Dynaflex® G2706 available from GLS Corporation, as well as other thermoplastic elastomers or silicone or urethane epoxies. The inner surface of the compressible portion 703 will thus conform to the surface of the sleeve 200 and any instruments inserted into the sleeve channels 213. The compressible portion 703 flexes to allow the outer member 215 to open, expand or uncompress when a medical instrument 500 is positioned within the outer member 215.

FIG. 7B illustrates a variation of the split ring 219a. (Like components between the embodiments of the split ring illustrated in FIGS. 7A and 7B have been designated with like reference numerals with an “a” suffix added to the reference numeral indicating the elements of the embodiment shown in FIG. 7B). The split ring 219a includes a compressible portion 703a that is molded around rigid dimples 705. The dimples project inward from the semi-rigid portion 701a. In a preferred form, the dimples and the semi-rigid portion are integrally molded of a relative hard plastic, and the compressible portion 703a is formed of a suitable material, such as a Kraton® polymer, as noted above. The dimples 705 and the compressible portion 707 act to seal the proximal portions 205, 217 to the endoscope 100 while allowing medical instruments 500 to pass between adjacent dimples 705, inside the compressible portion 703a and into the outer member 215. In the illustrated embodiment, one of the dimples generally corresponds with the split 707 in the ring 219a with the other dimples spaced apart by a distance corresponding to the spacing between the bonds 214 on the sleeve 200. In other embodiments, one or more of the dimples can be shaped (e.g., have a concave inner surface) and can back the compressible portion 703a at one or more locations that correspond to the position of the channels 213. Accordingly, the number of channels 213 and the number of dimples 705 need not correspond, nor do all of the dimples 705 need to have the same configuration.

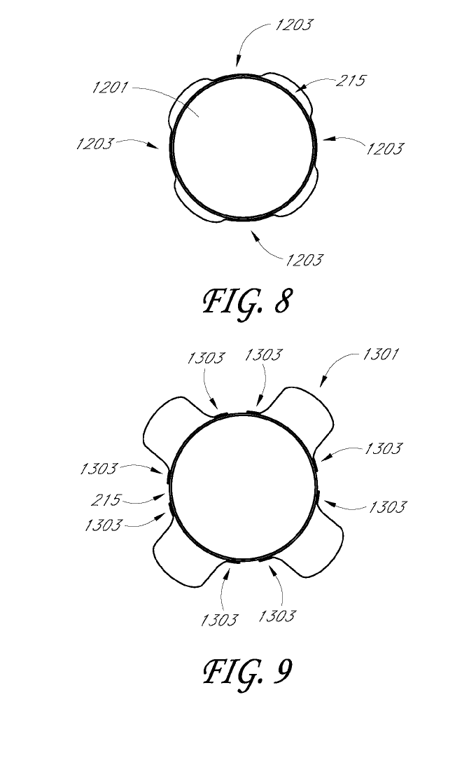

In this manner, the outer members 215 can be substantially sealed so as to prevent or substantially limit the flow of fluid, gas or other materials through the channels 213. In this preferred embodiment, the hollow member 201 is formed by forming or extruding an elastomeric material, such as silicone or a polymer, through a die or other similar device configured to form the hollow member 201. The outer member 215 can be integrally formed with the hollow member 201 during this process, or can be joined onto the hollow member 201 by a conversion process. In this later process (as shown in FIG. 8), the outer member 215 is joined to the hollow member 201 by inserting the hollow member 201 onto a tube 1201, and then placing the outer member 215 around the hollow member 201. The outer member 215 is then fused, adhered or otherwise coupled to the hollow member 201 at the bonds 214 to define one or more channels 213. In other embodiments, multiple outer members 215 can be attached to hollow member 201, as shown in FIG. 9, by coupling (e.g., fusing or adhering) the longitudinal side of each outer member 215 onto the hollow member 201.

In the preferred embodiments wherein the channeled sleeve 200 is integrally formed, the hollow member 201 and the outer member 215 are integrally formed by a dipping process, where the hollow member 201 is first formed by dipping a die of similar configuration to the cross-section shown in FIG. 4A into a liquid polymer, latex, polyurethane, or other bath and then removing and cooling the integrally formed channeled sleeve. In the preferred embodiments wherein the channeled sleeve 200 is formed by fastening the outer member 215 to the hollow member 201, the outer member 215 is formed by a separate dipping process than from the hollow member 201. The outer member 215 is formed onto the hollow member 201 through a similar dipping process using additional dies to form each channel.

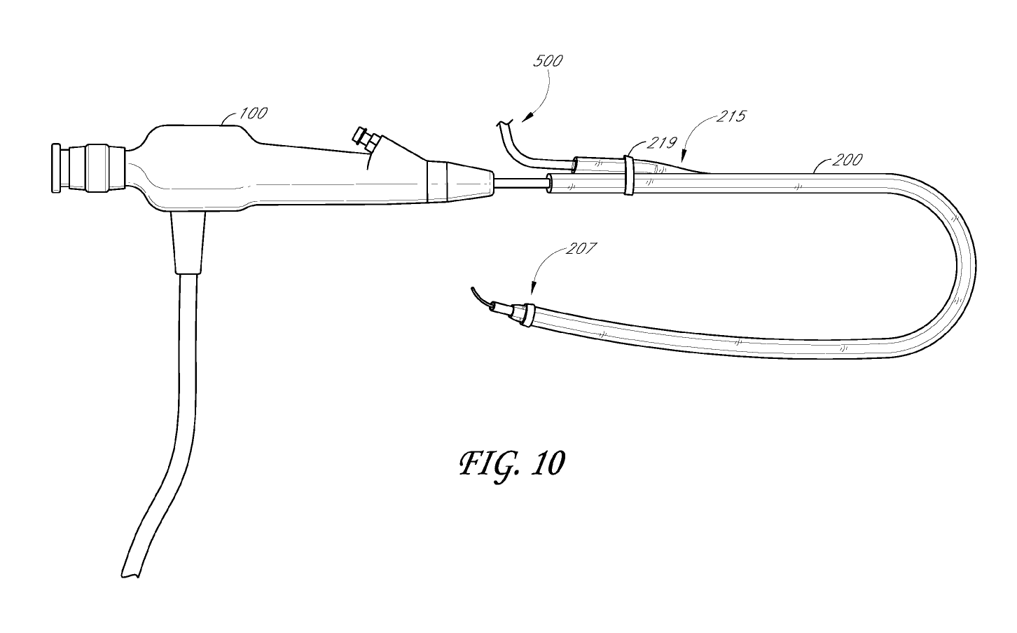

FIG. 10 illustrates a preferred method of using the channeled sleeve 200 wherein a split clip 219, as illustrated in FIGS. 7A and 7B, is attached to the endoscope 100 thereby securing the proximal end 205 of the channeled sleeve 200 to the endoscope 100, and forming a seal such that gases, fluids, and other materials are prevented from flowing proximally from the hollow member 201 and the outer member 215. For the split clip 219 as illustrated in FIG. 7A, the seal is formed when compressible portion 703 and the channeled sleeve 200 are sandwiched between the semi-rigid portion 701 of the split clip 219 and the endoscope 100. For the split clip 219a as illustrated in FIG. 7B, the seal is formed when compressible portion 703 and dimples 705 are sandwiched with the channeled sleeve 200 against the endoscope 100 by pressure from the semi-rigid portion 701.

As schematically shown in FIG. 10, a medical instrument 500 is inserted into the proximal portion 217 of the outer member 215, thereby creating a sealed opening by compressing/displacing the compressible portion 703 within the split clip 219. When positioning the medical instrument 500 within the outer member 215, the compressible portion 703 conforms around the medical instrument 500, thereby forming a seal around the medical instrument 500 and the endoscope 100, such that gases, fluids, and other materials are prevented from flowing proximally from the channeled sleeve 200, including the outer member 215.

Once the physician has inserted the medical instrument 500 into the proximal portion 217 of the outer member 215, the physician can then advance the medical instrument 500 through the outer member 215, which causes the outer member 215 to transition from the collapsed/compressed state (as illustrated in FIG. 4) to the expanded/uncompressed state (as illustrated in FIG. 5). The foregoing method is repeated for other medical instruments 500 to be placed in the channels 213 formed by the outer member 215 (or, in other embodiments, by other outer members 215).

In this preferred embodiment (as illustrated in FIG. 6A), the outer member 215 is open at the distal portion 216, such that when the band strip 207 is wrapped around the channeled sleeve 200, the outer member 215 is not occluded. In order to position the medical instrument at the surgical site, the physician axially traverses the medical instrument 500 through the outer member 215 and through the opening at the distal portion 216.

In some other preferred embodiments, an example of which was discussed above in connection with FIG. 6B, the outer member 215 is initially sealed. In such embodiments, the physician pushes the medical instrument 500 through the wall of the outer member 215 after the medical instrument 500 has reached the distal portion 216 of the outer member 215, thereby allowing the medical instrument 500 to be advanced into the surgical field.

An advantage of the channeled sleeve 200 is the ability to insert multiple medical instruments through the various channels 213 that are a part of the channeled sleeve 200, thereby providing multiple medical instruments with simultaneous access to the surgical site without having to replace or substantially reconfigure the physician's existing endoscope device 100. In this regard, endoscopic surgery (for example, a NOS or NOTES procedure) is made safer and more efficient because physicians need not repeatedly remove and insert various medical instruments 500 down the single channel 107 provided in the endoscope 100.

In the illustrated embodiment, as noted above, the physician may attach a vacuum or negative pressure source, which is readily available at the patient facility, to the proximal end 217 of the channels 213 to collapse the channels 213. By collapsing the channels 213, the physician may more freely remove or re-position the endoscope 100 and channeled sleeve 200 within the body. In other preferred embodiments, the physician may use channels 213 as a means for removing gas, fluid, tissue, or other materials from the body by applying a vacuum or negative pressure source to the channels 213. The physician may collect the extracted materials in a collection bag positioned at the proximal ends 217 of the channels 213 for further analysis and diagnosis.

In the illustrated embodiment, as noted above, the channeled sleeve 200 is easily removed (thus releasable) from the endoscope by removing the split clip 219 from the proximal portions 205, 217, and cutting the string or disengaging the band strip 207 from the distal portions 203, 216 by unwrapping the band strip 207 from the endoscope 100 and the hollow member 201. Some pulling force will be required to overcome the adhesion provide by the adhesive patch 212. The endoscope 100 is then withdrawn from the hollow member 201, and the single-use channeled sleeve 200 requires no cleaning because it is disposed after one use. In other preferred embodiments, the channeled sleeve 200 is removed from the endoscope 100 by cutting off the hollow member 201 or at least the distal portion thereof.

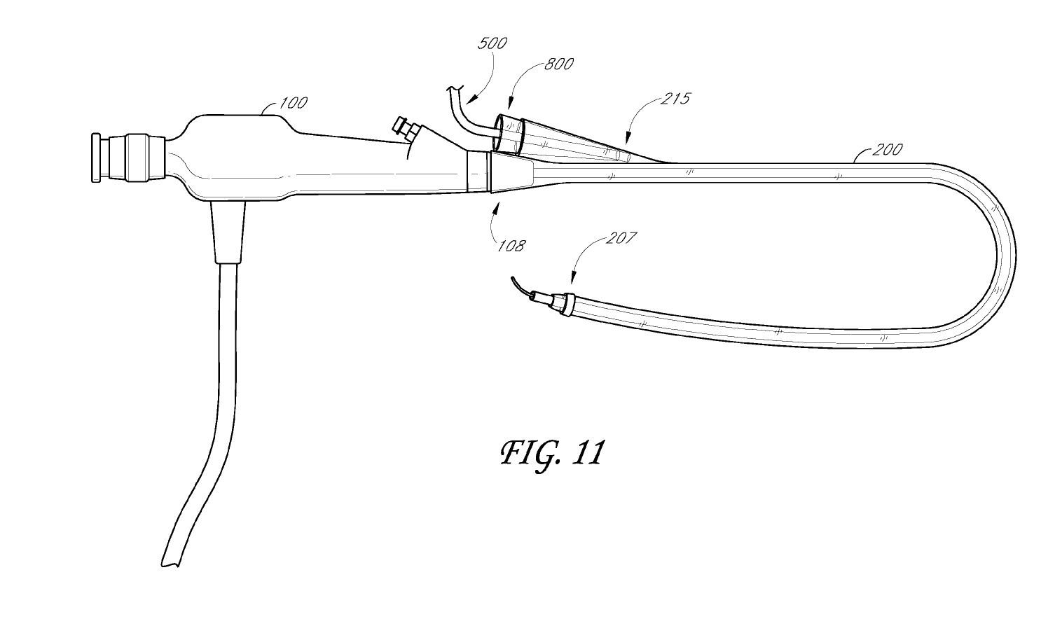

FIG. 11 illustrates another preferred method of using the channeled sleeve 200 wherein a proximal port 800 is inserted into the proximal portion 217 of the outer member 215, thereby forming a seal between the port 800 and the outer member 215 such that gas, fluid and other materials are inhibited from flowing proximally from the outer member 215. As illustrated, the channeled sleeve 200 preferably is positioned onto the distal housing portion 108. In this preferred embodiment, a split clip, split two piece clamp, or the like is not necessary to fix and seal the channeled member 200 to the endoscope because the tapered distal housing portion 108 forms a seal with the channeled sleeve 200 when the channeled sleeve 200 is positioned and stretched over the distal housing portion 108; however, in other embodiments a band, string, strip, split clip, or adhesive (as the foregoing are described herein), or other like fasteners, can be used to hold (as well as preferably seal) the proximal end 205 of the sleeve 200 to the distal housing portion 108. Additionally, the sleeve 200 need not extends onto the distal end 108 of the endoscopes hand piece 105. The clip, band or like fastener can engage the proximal portion 104 of the endoscope body rather than the hand piece 105.

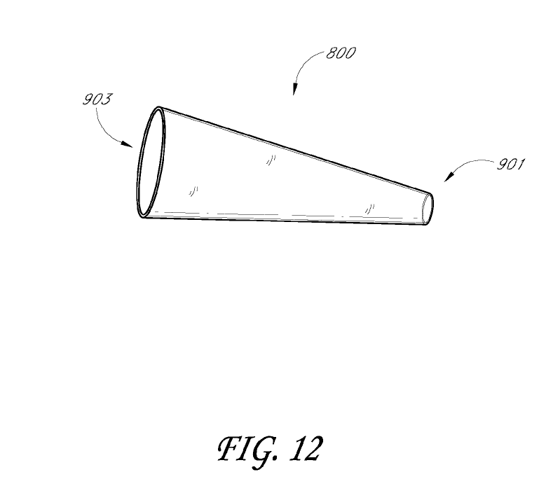

FIG. 12 illustrates the proximal port 800 as a substantially cone shaped or conical device wherein the narrow distal end 901 of the proximal port 800 is configured to be inserted into the proximal opening 217 of the outer member 215, and the wide proximal end 903 of the proximal port 800 is configured to receive a medical instrument 500. An internal seal (such as a septum seal or duck bill valve, as both are disclosed in, for example, U.S. Pat. No. 5,209,737 and U.S. Pat. No. 6,997,931, and both are hereby incorporated by reference in their entirety) is provided within the port 800, thereby preventing lost of insufflation at the distal end of the channeled sleeve 200 when the port 800 is inserted into the outer member 215. When a medical instrument 500 enters and traverses the channel 213 within the outer member 215 the internal seal maintains the seal around the medical instrument 500, and prevents lost of insufflation at the distal end of the channeled sleeve 200.

In use, a physician inserts the medical instrument 500 through the proximal port 800 and into the proximal portion 217 of the outer member 215. As the physician axially traverses the medical instrument 500 through the outer member 215, the outer member 215 transitions from a collapsed/compressed state (as illustrated in FIG. 4) to an expanded/uncompressed state (as illustrated in FIG. 5). Advancement of the instrument into the surgical field is accomplished in the manners described above. Additionally, a proximal port 800 can be used with each channel proximal open 217 to seal the proximal ends of the channels 213. Alternatively, the proximal ends of the channels 213 can be initially closed and the distal end 901 of the port 800 can pierce the outer member 215 to be inserted into the corresponding channel 213. (An opening into the channel 213, through which the distal end 901 can be inserted, can be formed in other manners as well.) In either case, the ports 800 can be a reusable item, which is cleaned and/or sterilized after each use, or be a disposable item, which is discarded along with the channeled sleeve after use.

In the above-described embodiments, the one or more channels 213 of the member 200 can be formed by a single outer member 215 with multiple (e.g., four) longitudinally extending bonds that join the outer member 215 to the hollow member 201, as illustrated in FIG. 8. Alternatively, the one or more channels 213 can be formed using multiple outer members 215. Each outer member can have a strip-like shape before attachment to the hollow member 214 (e.g., the embodiment illustrated in FIG. 9) or can have a tubular-like shape of a cross-section size larger or smaller than that of the hollow member 201.

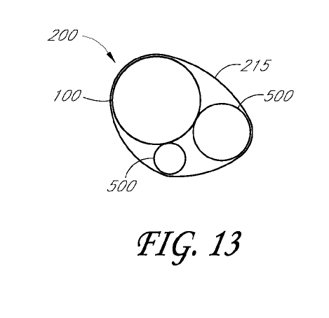

FIG. 13 illustrates another embodiment of the channeled sleeve. In this embodiment, multiple channels 213 are replaced by a single channel 213 that is configured to allow multiple medical instruments 500 to axially traverse the outer member 215 as illustrated in FIG. 13. In this embodiment, the channeled sleeve 200 comprises a single longitudinally extending bond between the hollow member 201 and the outer member 215.

In other preferred embodiments of the present invention, the band strip 207 is substituted with a band structure that is positioned at the distal portions 203, 216 and that is separate from the hollow member 201. According to this preferred embodiment, after the channeled sleeve 200 is positioned onto the endoscope 100, the channeled sleeve 200 is secured to the endoscope 100 by releasably positioning the band structure over the channeled sleeve 200 to releasably engage the endoscope 100. The band structure can be elastic and stretched over the endoscope and distal portion 203 of the sleeve, or can be wrapped around the sleeve in a manner similar to that described above.

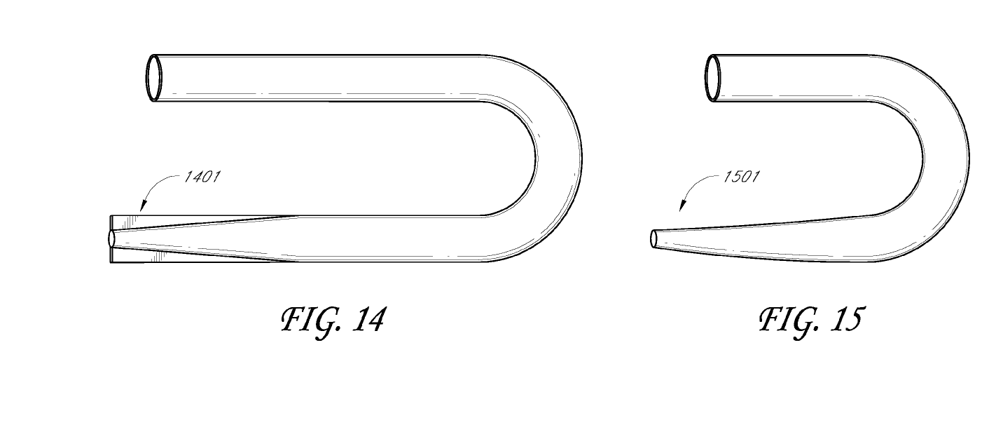

In other preferred embodiments, the band strip 207 is replaced by other securing means. For example, in some preferred embodiments the channeled sleeve 200 is secured to the endoscope 100 by configuring the hollow member 201 to comprise a tapered distal portion 1401, 1501, as illustrated in FIGS. 14 and 15. In these foregoing preferred embodiments, the channeled sleeve 200 is releasably secured to the endoscope 100 when the endoscope 100 is positioned in the tapered distal portion 1401, 1501. Because the diameter of the tapered distal portions 1401, 1501 is configured to be smaller than the diameter of the endoscope 100, a radial inward pressure is applied by the tapered distal portion 1041, 1501 to engage the endoscope 100 thereby releasably securing the channeled member 200 to the endoscope 100. The tight fit caused by stretching the materials of the hollow member' distal end 203 as the endoscope 100 is inserted also forms a sufficient seal between the endoscope 100 and the hollow member 201.

In other preferred embodiments, the band strip 207 is substituted with a split clip, or the like, configured to releasably secure and seal the channeled sleeve 200 to the endoscope 100. In still other embodiments, an adhesive is placed on the interior surface of the hollow member 201 so as to releasably secure and seal the channeled sleeve 200 to the endoscope 100.

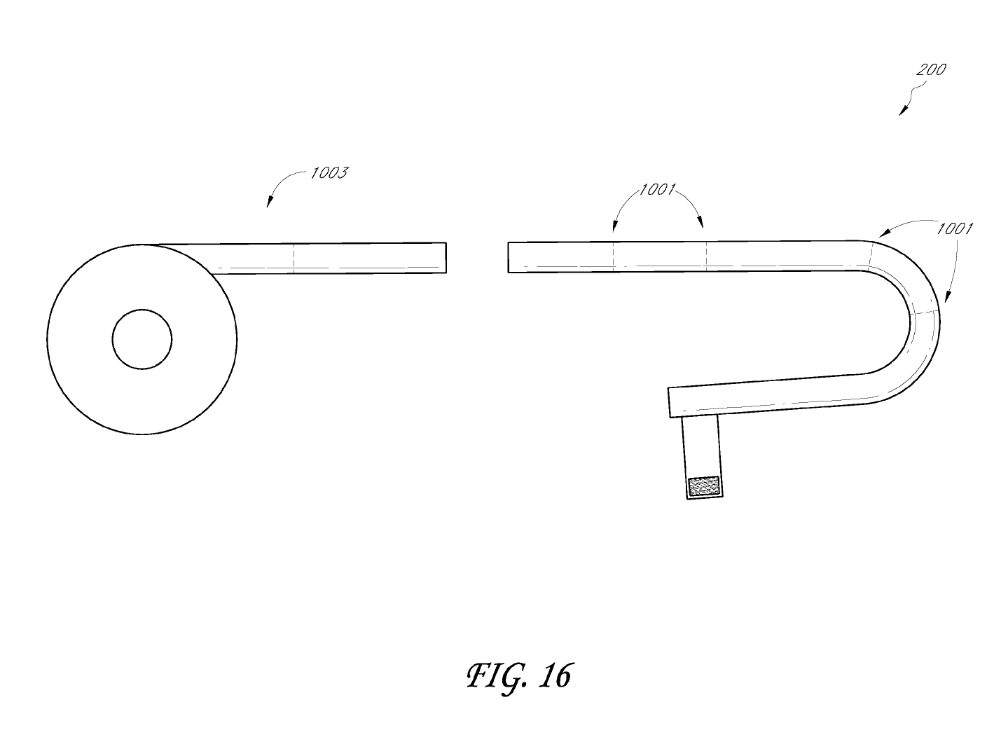

In some embodiments, the length of the channeled sleeve 200 is adjustable to suit the working length of the endoscope 100. In accordance with this preferred embodiment, FIG. 16 illustrates a channeled sleeve 200 comprising perforated areas 1001 for adjusting the working length of the channeled sleeve 200. In some preferred embodiments, the channeled sleeve 200 is stored in a spindle 1003 wherein a physician can withdraw a channeled sleeve 200 of a certain length and then remove excess portions thereof by tearing the channeled sleeve 200 along a perforated area 1001.

The channeled sleeve 200, or at least the channels 213 (and thus the outer member 215), has a length that is at least substantially co-extensive with the working length of the endoscope 100. In some preferred embodiment, the working length of the channeled sleeve 200 is the distance from the endoscope distal portion 103 along the length of the shaft of the endoscope 100 to a point that remains outside the body in use. In still other preferred embodiments, the length of the channeled sleeve 200 is the full length of the endoscope 100. In this manner, access to the proximal ends of the channels 213 is maintained even when the endoscope 100 is fully positioned within the patient's body.

In some preferred embodiments, the channeled sleeve 200 comprises openings to allow the existing markings 106 on the endoscope 100 to be visible to the physician. In other preferred embodiments, the channeled sleeve 200 comprises windows to allow the existing markings 106 on the endoscope 100 to be visible to the physician. In still other preferred embodiments, the channeled sleeve 200 is translucent or transparent to allow the markings 106 on the endoscope 100 to be visible to the physician. In other preferred embodiments, the channeled sleeve 200 comprises markings to indicate the position of the channeled sleeve 200 within the body. In some preferred embodiments, the channeled sleeve 200 comprises an exterior surface that is substantially smooth to substantially reduce or mitigate rough areas and/or sharp edges of the endoscope 100.

Although the inventions have been disclosed in the context of a certain preferred embodiments and examples and in the context of use with an endoscope, it will be understood by those skilled in the art that the present inventions extend beyond the specifically disclosed embodiments to other alternative embodiments and/or uses of the inventions and obvious modifications and equivalents thereof. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above.

1. A single-use sleeve for an endoscope, which has an elongated body having a working length that terminates at a distal end, the sleeve comprising:
a flexible elongated generally cylindrical hollow member having an inner size and cross-sectional shape that generally matches the cross-sectional shape and size of the endoscope elongated body at least along the working length such that the endoscope fits within the hollow member, the hollow member having a wall thickness which is at least an order of magnitude smaller than the inner size of the hollow member;
wherein the hollow member comprises a distal portion configured for attachment to a distal portion of the endoscope to substantially secure the hollow member to the endoscope; and
at least one flexible elongated outer member attached to the hollow member to define at least one elongated channel, the channel having a length that is at least substantially equal to the working length of the endoscope's elongated body, the channel having a sealed proximal end, the channel being sized so as to receive a medical instrument.

2. The single-use sleeve of claim 1, wherein the hollow member lacks columnar strength.

3. The single-use sleeve of claim 1, wherein the inner size is no smaller than 20 mm.

4. The single-use sleeve of claim 1, wherein the hollow member and the outer member comprise a surface, the surface being substantially smooth to substantially reduce rough or sharp edges at least along the working length of the endoscope.

5. (canceled)

6. (canceled)

7. (canceled)

8. (canceled)

9. The single-use sleeve of claim 1, wherein the outer member is substantially collapsible about the hollow member.

10. The single-use sleeve of claim 1, wherein the outer member is configured to have a collapsed position and an expanded position.

11. The single-use sleeve of claim 1, wherein the length of the channel is adjustable.

12. The single-use sleeve of claim 1, wherein the channel comprises at least one perforated area configured to adjust the length of the channel.

13. The single-use sleeve of claim 1, wherein the hollow member is at least as flexible as the endoscope.

14. (canceled)

15. The single-use sleeve of claim 1, wherein the hollow member and the outer member further comprise a plurality of windows to allow indicia on the endoscope to be visible.

16. The single-use sleeve of claim 1, wherein at least one of in the hollow member and outer member are sufficiently translucent or transparent to allow indicia on the endoscope to be visible.

17. The single-use sleeve of claim 1, wherein the attachment device is a band configured to extend about the hollow member.

18. The single-use sleeve of claim 1, wherein the attachment device is a split clip, the clip configured to extend about at least a substantial portion of the hollow member against the endoscope.

19. The single-use sleeve of claim 18, wherein the clip comprises at least one groove area configured to allow axial movement of the medical instrument through the groove area.

20. The single-use sleeve of claim 1, wherein the attachment device includes a tab that extends outwardly, away from a longitudinal axis of the sleeve, the tab including an adhesive layer on at least one side of the tab.

21. The single-use sleeve of claim 20, wherein the tab is attached to the hollow member.

22. The single-use sleeve of claim 1, wherein the channel is sealed along it length.

23. An endoscopic system comprising:
an endoscope having a body configured to be inserted into patient, the body housing an optical element and an instrument channel, both the optical element and the instrument channel terminating at a distal end of the body; and
at least one auxiliary channel attached to the exterior of the body, the auxiliary channel being sized to receive at least one medical instrument.

24. A method of performing endoscopic surgery comprising:
inserting an endoscope into a flexible sleeve having at least one external channel, the sleeve having a radial thickness substantially less than that of the endoscope;
attaching the sleeve onto a distal end of the endoscope such that the sleeve moves with the endoscope at least when the endoscope is advanced;
introducing the endoscope and sleeve into a patient body and simultaneously advancing the distal ends of the endoscope and sleeve to a target site;
inserting a medical instrument through a seal into the channel of the sleeve;
advancing the instrument through the channel until the distal end of the instrument extends beyond a distal end of the endoscope into the target site; and
using the instrument to perform a medical procedure.

25. The method of claim 24, wherein the endoscope and sleeve are inserted into the patient body through a natural orifice.
